(19) United States
(12) Patent Application Publication
(10) Pub. No.: US 2010/0008944 A1
(43) Pub. Date: Jan. 14, 2010

(54) HERPES SIMPLEX VIRUS MUTANT AND USES THEREFORE

(75) Inventors: David Knipe, Aubumdale, MA (US); Timothy Dudek, Brookline, MA (US)

Correspondence Address: EDWARDS ANGELL PALMER & DODGE LLP, P.O. BOX 55874, BOSTON, MA, 02205, US

(73) Assignee: PRESIDENT AND FELLOWS OF HARVARD COLLEGE, Cambridge, MA (US)

(21) Appl. No.: 11/989,687

(22) PCT Filed: Jul. 26, 2006
(86) PCT No.: PCT/US2006/029185

§ 371 Date: Sep. 23, 2009

Related U.S. Application Data
(60) Provisional application No. 60/703,537, filed on Jul. 29, 2005.

(51) Int. Cl. A61K 39/245; C12N 7/01; C12N 5/10; C07H 21/00; C12N 15/63; A61P 31/12

(52) U.S. Cl. 424/199.1; 435/235.1; 435/325; 536/23.72; 435/320.1

(57) ABSTRACT

The invention generally provides therapeutic and prophylactic compositions that include a replication defective mutant HSV-2 virus having a mutation in a viral host shut-off protein, a herpes simplex virus having a mutation in a viral host shut-off protein and two additional mutations that render the virus replication defective, and related methods.

### CROSS-REFERENCE TO RELATED APPLICATION

This application claims the benefit of U.S. Provisional Application No. 60/703,537, which was filed on Jul. 29, 2005, the contents of which are incorporated herein by reference in their entirety.

### BACKGROUND OF THE INVENTION

Herpes simplex viruses types 1 and 2 are ubiquitous human pathogens affecting nineteen percent of the adult U.S. population. The herpes virus is an enveloped virus that contains a 152 kb dsDNA genome that includes eighty-four open reading frames. The primary site of herpes infection is the epithelium and the virus also undergoes replication there. When viral particles are released from the epithelium, they can infect local sensory neurons. The viral particle is transported from the sensory axon back to the cell body of the sensory neuron where it can establish a latent infection.

Human infection by these herpes viruses typically results in lifelong latent infections that periodically give rise to clinical lesions or asymptomatic viral shedding. Herpes viruses are a major cause of sexually transmitted disease for which no adequate therapies exist. Because transmission of the virus can occur even in the absence of symptoms, public health measures to control the sexual transmission of the virus have been largely ineffective. In addition, chronic infection with the virus lowers immune function and increases the probability that an infected individual will acquire human immunodeficiency virus (HIV).

Herpes infections can also be transmitted from a mother to her infant during childbirth. The resulting neonatal infections have a fifty percent mortality rate and even when the neonate survives the infection, neurological sequelae are common. Better methods of treating and preventing herpes infection are urgently required.

### SUMMARY OF THE INVENTION

The invention generally provides therapeutic and prophylactic compositions that include a replication defective mutant HSV-2 virus having a mutation in the viral host shut-off protein. In addition, the invention provides therapeutic and prophylactic compositions that include a herpes simplex virus (e.g., HSV-1 or HSV-2) having a mutation in a viral host shut-off protein and two additional mutations in genes required for viral replication. The invention further features therapeutic methods related to these viruses. In particular, the invention features a replication defective HSV-2 virus having an insertion within the UL41 gene locus (i.e., an HSV-2 dl5-29-41LacZ mutant).

In one aspect, the invention generally features an isolated herpes simplex virus 2 (HSV-2) containing an alteration in a UL41 nucleic acid sequence, where the alteration increases the immunogenicity of the virus, and at least one additional alteration in a nucleic acid sequence required for viral replication. In one embodiment, the alteration in UL41 is a missense mutation, insertion or deletion, such as an insertion that interferes with the expression of a virion host shut-off protein. In another embodiment, the alteration in UL41 is the deletion of at least a portion of UL41 or is the deletion of UL41. In yet another embodiment, the additional alteration is an alteration in a UL5 or UL29 nucleic acid sequence, such as a missense mutation, insertion or deletion (e.g., the deletion of UL5 and/or UL29) that renders the virus replication defective.

In another aspect, the invention generally features an isolated herpes simplex virus (e.g., HSV-1 or HSV-2) comprising an alteration in a UL41 nucleic acid sequence, where the alteration increases the immunogenicity of the virus, and at least two additional alterations in nucleic acid sequences required for viral replication.

In another aspect, the invention features a host cell (e.g., a mammalian cell, such as a human cell) containing a virus of a previous aspect. In one embodiment, the cell is a dendritic cell or other professional antigen presenting cell.

In another aspect, the invention features an isolated HSV-2 nucleic acid molecule containing an alteration in a UL41 nucleic acid sequence and at least one additional alteration in a gene that is required for replication. In one embodiment, the alteration in UL41 is a missense mutation, deletion or insertion, such as an insertion that interferes with the expression of a virion host shut-off protein. In another embodiment, the alteration in UL41 is the deletion of at least a portion of UL41 or the deletion of UL41. In another embodiment, the additional alteration is an alteration in a UL5 or UL29 nucleic acid sequence, such as a missense mutation, insertion or deletion (e.g., the deletion of UL5 or UL29 or the deletion of UL5 and UL29). In another embodiment, the nucleic acid molecule comprises a heterologous sequence. In yet another embodiment, the heterologous sequence encodes a protein capable of eliciting an immune response in a host.

In another aspect, the invention generally features an isolated herpes simplex virus (e.g., HSV-1 or HSV-2) comprising an alteration in a UL41 nucleic acid sequence, where the alteration increases the immunogenicity of the virus, and at least two additional alterations in nucleic acid sequences required for viral replication.

In another aspect, the invention features a vector containing a nucleic acid molecule of a previous aspect. In one embodiment, the vector is a viral vector (e.g., an adenoviral vector, adeno-associated viral vector, retroviral vector, lentiviral vector, alphaviral vector, and herpes virus vector).

In yet another aspect, the invention features a vaccine containing an HSV-2 virus (e.g., a virus of any previous aspect) in a pharmaceutically acceptable excipient, where the virus comprises an alteration in a nucleic acid sequence that encodes a virion host shut-off protein and an additional alteration that renders it replication defective.

In a related aspect, the invention features a vaccine containing an HSV-2 nucleic acid molecule in a pharmaceutically acceptable excipient, where the nucleic acid molecule comprises an alteration in a UL41 nucleic acid sequence and an additional alteration that renders it replication defective. In one embodiment, the alteration is a missense mutation, insertion (e.g., an insertion that interferes with the expression of a virion host shut-off protein) or deletion, such as the deletion of at least a portion of UL41 or the deletion of UL41. In another embodiment, the additional alteration is an alteration (e.g., a missense mutation, insertion or deletion) in a UL5 or UL29 nucleic acid sequence. In yet another embodiment, the alteration is the deletion of UL5 or UL29 or is the deletion of UL5 and UL29. In another embodiment, the composition further comprises an adjuvant. In yet another embodiment, the composition elicits a cellular or a humoral immune response when injected into a subject (e.g., a mammal, such as a human).

A vaccine containing a cell (e.g., a mammalian cell, such as a human dendritic cell or professional antigen presenting cell) infected with an HSV-2 virus in a pharmaceutically acceptable excipient, where the HSV-2 virus comprises an alteration (e.g., a missense mutation, insertion or deletion) in UL41 and an additional alteration that renders the virus replication defective. In one embodiment, the alteration in UL41 is an insertion that interferes with the expression of a virion host shut-off protein. In another embodiment, the alteration in UL41 is the deletion of at least a portion of UL41 or is the deletion of UL41. In another embodiment, the additional alteration is an alteration (e.g., a missense mutation, insertion or deletion) in a UL5 or UL29 nucleic acid sequence. In one embodiment, the additional alteration is the deletion of UL5 or UL29 or is the deletion of UL5 and UL29. In another embodiment, the cell is derived from a subject (e.g., a mammal, such as a human). In yet another embodiment, the cell (e.g., a dendritic cell) is present in a blood sample from the subject. In yet another embodiment, the cell is cultured and infected ex vivo.

In another aspect, the invention features an immunogenic composition containing an HSV-2 virus in a pharmaceutically acceptable excipient, where the virus comprises an alteration (e.g., a missense mutation, insertion or deletion) in a UL41 nucleic acid sequence and an additional alteration that renders the virus replication defective. In one embodiment, the alteration in UL41 is an insertion that interferes with the expression of a virion host shut-off protein. In another embodiment, the alteration in UL41 is the deletion of at least a portion of UL41 or is the deletion of UL41. In yet another embodiment, the additional alteration is an alteration (e.g., a missense mutation, insertion or deletion) in a UL5 or UL29 nucleic acid sequence, such as the deletion of UL5 and/or UL29. In yet another embodiment, the composition enhances or induces an immune response (e.g., a cellular or humoral immune response) when injected into a subject (e.g., a mammal, such as a human).

In another aspect, the invention features an immunogenic composition containing a herpes simplex virus in a pharmaceutically acceptable excipient, where the virus comprises an alteration (e.g., a missense mutation, insertion or deletion) in a UL41 nucleic acid sequence and two additional alterations that render the virus replication defective.

In a related aspect, the invention features a method for treating a herpes infection in a subject (e.g., a mammal, such as a human). The method involves administering to the subject a vaccine of any previous aspect, where the vaccine treats or prevents a herpes infection, such as a herpes infection associated with a herpetic lesion, stromal keratitis, meningitis, or encephalitis. In one embodiment, the method further involves administering an anti-viral agent (e.g., acyclovir or valcyclovir). In another embodiment, the method enhances an immune response in a subject; reduces the number or frequency of herpes outbreaks in a subject; or reduces viral shedding in a subject.

In a related aspect, the invention features a method of preventing a herpes infection in a subject, the method involves administering to the subject a vaccine composition of any previous aspect, where the vaccine prevents a herpes infection. In one embodiment, the method prevents the infection of a cell, such as an epithelial or a neuronal cell, or prevents the establishment of a latent infection in a subject (e.g., a mammal, such as a human) infected with a herpes virus.

In another aspect, the invention features a kit for treating or preventing a herpes viral infection in a subject, the kit containing a herpes vaccine of any previous aspect. In one embodiment, the herpes virus is replication defective. In another embodiment, the kit further contains instructions for administering the herpes virus to a subject.

### BRIEF DESCRIPTION OF THE DRAWINGS

FIG. 1 is a graph showing the results of an Elisa assay for HSV-2 IgG antibodies generated by mice immunized with the dl5-29 double deletion viral vaccine (light bars) or with the triple mutant dl5-29-41LacZ vaccine (dark bars). Mice received three injections each of which contained 10⁶ pfu of virus. The HSV-2 IgG was analyzed in serum obtained one week after the second (2°) and third (3°) injections.

FIGS. 2A and 2B are a schematic diagram and a photographic panel, respectively. FIG. 2A illustrates the scheme required to carry out the ELISpot (enzyme-linked immunospot) assay. FIG. 2B provides a photograph of exemplary results obtained using the ELSpot assay.

FIG. 3 is a graph showing the results of an ELISpot analysis of the frequency of IFN-γ-secreting splenocytes in a heterogenous population of splenocytes isolated from mice that were immunized (as described herein) with 10⁵ pfu of a dl5-29 double deletion viral vaccine (light bars) or with a triple mutant dl5-29-41LacZ vaccine. Results are shown for splenocytes isolated after the second viral immunization (2°) and after the third (3°) viral immunization.

FIG. 4 is a graph showing the results of an ELISpot analysis of the frequency of IFN-γ-secreting splenocytes in a heterogenous population of splenocytes isolated from mice that were immunized (as described herein) with 10⁶ pfu of a dl5-29 double deletion viral vaccine (light gray bars), with a triple mutant dl5-29-41LacZ vaccine (dark gray bars), or with a mock vaccine (white bars). Results are shown for splenocytes isolated after the primary immunization and after the second immunization.

FIG. 5 is a graph showing the HSV-2 total IgG titer present in mice that received two immunizations with 10⁴, 10⁵, or 10⁶ pfu each of a dl5-29 double deletion viral vaccine (light gray bars) or a triple mutant dl5-29-41LacZ vaccine (dark gray bars). These mice were subsequently challenged by infection with a pathogenic HSV-2 virus.

FIG. 6 is a graph showing the survival of mice immunized with 10⁴ pfu of a dl5-29 double deletion viral vaccine (DL529), a triple mutant dl5-29-41LacZ vaccine (VHS), or a mock vaccine (mock) and then challenged with a wild-type HSV-2 virus (50LD₅₀ HSV-2 G).

FIG. 7 is a graph showing the survival of mice immunized with 10⁵ pfu of a dl5-29 double deletion viral vaccine (DL529), a triple mutant dl5-29-41LacZ vaccine (VHS), or a mock vaccine (mock) and then challenged with a wild-type HSV-2 virus (50LD₅₀ HSV-2 G).

FIG. 8A is a graph showing the survival of mice immunized with 10⁶ pfu of a dl5-29 double deletion viral vaccine (DL529), a triple mutant dl5-29-41LacZ vaccine (VHS), or a mock vaccine (mock) and then challenged with a wild-type HSV-2 virus (50LD₅₀ HSV-2 G).

FIG. 8B is a graph showing the survival of mice immunized with 10⁵ or 10⁶ pfu of a dl5-29 double deletion viral vaccine (DL529), a triple mutant dl5-29-41LacZ vaccine (VHS), or a mock vaccine (mock) and then challenged with a wild-type HSV-2 virus (50LD₅₀ HSV-2 G).

FIG. 9 is a graph showing viral shedding in mice that were immunized with 10⁵ or 10⁶ pfu of a dl5-29 double deletion viral vaccine (DL529), a triple mutant dl5-29-41LacZ vaccine (VHS), or a mock vaccine (mock). The levels of viral shedding is shown in pfu per ml on the y-axis.

FIG. 10 shows protein expression in Vero cells infected with a wild-type virus (wt), a dl5-29 double deletion virus, a triple mutant dl5-29-41LacZ virus, or mock infected. Protein expression was analyzed at four, six, and eight hours post-infection.

FIGS. 11A-11D are schematic diagrams showing the genome structure of wild-type and mutant HSV-2 strains. FIG. 11A shows the genome structure of the wild-type virus. Boxes represent repeated sequences in the viral genome, and lines represent the unique sequences. FIG. 11B is a schematic of expanded regions showing sequence features in the vicinity of the UL5 and UL29 viral genes. Boxes indicate the locations and orientations of ORFs. Arrows indicate the start sites and direction of transcription. FIG. 11C shows the genomic locations of the dl5 and dl29 deletion mutations. Numbers corresponding to base pairs in the HSV-2 strain HG-52 sequence. FIG. 11D shows the sequence coordinates of the HSV-2 UL5 gene (left) and HSV-1 UL29 gene (right) transformed into Vero cells to make V529 cells.

FIGS. 12A and 12B are schematic diagrams showing the sequence of the dl5 deletion site in dl5-29 viral DNA and the sequence of the dl29 deletion site in dl5-29 viral DNA, respectively.

FIG. 13 is a schematic diagram showing the genome structure of the mutant dl5-29-41L strain in the HSV-2 strain HG-52 sequence. As shown in the diagram LacZ insertion into UL41 extends from base 92342 to 92860 in the HSV-2 strain HG52.

FIG. 14 shows the amino acid and nucleic acid sequence of wild-type UL41 in HSV-2 strain 333.

### DETAILED DESCRIPTION OF THE INVENTION

### Definitions

By “UL41” is meant a herpes gene encoding a virion host shut-off (VHS) protein. The sequence of UL41 HSV-2 strain 333 UL41 is provided at GenBank Accession No. AF007816. The sequence of HSV-2 strain HG-52 complete genome is provided at GenBank accession No. Z86099, which is hereby incorporated by reference.

By “replication defective” is meant having a reduced ability or lacking the ability to undergo replication or lacking the ability to generate viable progeny.

By “immunogenic composition” is meant an agent that induces an immune response in a subject.

By “alteration” is meant any change in a nucleic acid or amino acid sequence relative to a reference sequence.

By “ameliorate” is meant decrease, suppress, attenuate, diminish, arrest, or stabilize the development or progression of a disease.

By “disease” is meant any condition or disorder that damages or interferes with the normal function of a cell, tissue, or organ.

By “enhance an immune response” is meant increase an immune response in a subject by at least 10% relative to a control subject where the immune response was not enhanced. Preferably, the immune response is increased by 25%, 50%, 75% or 100%.

By “immune response” is meant any cellular or humoral response against an antigen.

By “isolated nucleic acid molecule” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences.

By “polypeptide” is meant any chain of amino acids, regardless of length or post-translational modification.

By “subject” is meant a mammal, such as a human patient or an animal (e.g., a rodent, bovine, equine, porcine, ovine, canine, feline, or other domestic mammal).

A “therapeutically effective amount” is an amount sufficient to effect a beneficial or desired clinical result.

By “treat” is meant stabilize, reduce, or ameliorate the symptoms of any disease or disorder.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### METHODS OF THE INVENTION

As reported in more detail below, the invention provides methods and compositions useful for the treatment or prevention of a herpes infection in a subject. In general, this invention is based on the discovery that a replication defective herpes virus containing a disruption of the UL41 gene, which encodes a virion host shut-off (VHS) protein, was significantly more effective in generating an immune response in vivo than was a replication defective virus expressing the VHS protein. Accordingly, the invention provides therapeutic vaccines and prophylactic vaccines that contain a herpes virus or nucleic acid molecule having a mutation in UL41 (e.g., dl5-29-41LacZ); methods of using such vaccines for the treatment or prevention of herpes infections; combination therapeutics that provide for the administration of such vaccines in combination with any standard anti-viral therapeutic; and related kits.

### Replication Defective HSV-2 Viruses

HSV-2 replication defective strains having a mutation in an HSV-2 UL41 gene are useful as vaccines for eliciting a protective immune response or for enhancing an immune response against HSV-2. The replication defective mutants of the invention are not limited to HSV-2 viruses containing mutations in the UL5 or UL29 genes. Rather, any HSV-2 virus that is incapable of replication may be useful in the methods of the invention. Genes required for replication are known in the art. See, for example, HSV-2 strain HG-52 complete genome (GenBank accession No. Z86099).

Replication defective herpes viruses useful in the methods of the invention contain at least one mutation in a nucleic acid sequence required for replication. Desirably, an HSV-2 replication defective mutant contains at least two mutations in genes that are required for replication, at least one mutation of which renders the virus replication defective. Preferably, two or more of the mutations independently render the virus replication defective, such as where two or more of the genes encoding UL5 and UL29 are both mutated. See, for example, U.S. Patent Publication No. 20020009462 and DaCosta et al. (J. Virology 74:7963-7971, 2000).

Examples of these viruses include, but are not limited to, strains in which a mutation in a UL29 gene is introduced into the genome of a virus that contains an existing mutation in a UL5 gene. Conversely, a virus strain can be constructed in which a mutation in the UL5 gene is introduced into the genome of a virus that contains an existing mutation in a UL29 gene. In order to replicate such viruses, a cell line is generated that is capable of expressing both the wild-type genes corresponding to the mutated genes. The procedures for the generation of cell lines that express more than one herpes virus gene are known in the art and are similar to those described herein.

Alternatively, a herpes simplex virus may contain a mutation in one or more of an α or immediate-early (IE) genes, a β or early (E) genes, and γ or late (L) genes. In one embodiment, a herpes simplex virus contains a mutation in ICP4, a gene that is required for all delayed-early and late gene transcription. The expression of β genes is dependent on newly synthesized α gene products, in particular, the ICP4 and ICP27 proteins. Many of the β proteins are involved in viral DNA replication. Viral DNA synthesis, in concert with ICP4, ICP27, and ICP8, triggers the expression of γ gene products, most of which are viral structural proteins that are involved in virion assembly and maturation. ICP27 stimulates expression of some early genes and transcription of at least some late viral genes. The early DNA-binding protein, ICP8, also stimulates late gene transcription. Replication defective herpes viruses may include a mutation in any one or more of an immediate early or early gene. For example, such viruses may contain mutations in any one or more of ICP8, ICP4, or ICP27.

### Methods for Generating a Mutant HSV-2 Virus

HSV-2 viruses having a mutation in a UL41 gene are useful in the methods of the invention. Also useful are HSV-2 viruses that contain mutations in UL41 and in a second gene that renders the virus replication defective (e.g., a mutation in UL5 or UL29). Desirable mutations include nonsense, deletion, insertion, or point mutations. Methods for producing a preferred mutation in a herpes viral gene are known in the art. Briefly, any desired mutation may be generated as follows. Plasmids are constructed which comprise DNA encoding the appropriate mutation, flanked by DNA that will undergo homologous recombination. The plasmid is cotransfected into cells, such as animal cells, that contain the herpes viral genome into which the mutation is to be inserted. The mutation is then introduced into the parental genome by recombination during viral DNA replication. Progeny viruses are screened for the presence of the mutation using techniques known in the art. For example, progeny virus can be screened for their ability to replicate only in a cell line expressing a wild-type complementing copy of the mutated gene that expresses the gene that is essential for virus replication. Viruses having a desired mutation can also be identified, for example, by Southern blot hybridization, Western blotting, immunofluorescence, polymerase chain reaction, or any other method known in the art.

The replication defective and UL41 mutant viruses employed herein can be derived from the HSV-2 virus as described herein. The virus can be rendered replication defective by effectively mutating a gene or genes encoding one or more proteins required for completing the replication cycle (e.g., UL5 or UL29).

### Therapeutic and Prophylactic Vaccines

A prophylactic viral vaccine is typically used to induce extended (e.g., lifetime) protection against infection of a subject with a herpes virus, to prevent the establishment of a latent infection in a subject, or to prevent the recurrence of symptoms associated with an existing herpes infection in a subject. Therapeutic viral vaccines are used to enhance an immune response against a herpes virus in a subject. Desirably, a therapeutic vaccine reduces or ameliorates the symptoms associated with a herpes outbreak (e.g., occurrence of lesions), reduces or eliminates the risk of recurrence of an outbreak, or prolongs the interval between outbreaks. Desirably, the prophylactic and therapeutic vaccines of the invention are free of both initial and long term side effects. The vaccine should induce both protective humoral antibodies and/or cell-mediated immunity. Live virus vaccines should be incapable of spreading from vaccines to non-vaccinated individuals, and should not be capable of latent infection in the vaccine. In one embodiment, the vaccine contains a mutant herpes virus that is incapable of acquiring a wild-type version of a mutant gene. In other embodiments, the probability of reversion is so low as to be negligible.

Mutant herpes viruses described herein satisfy these criteria. Specifically, vaccines according to the invention should have at least one or more of the following properties: they should be viable and yet be effectively incapable of producing viable progeny virus in the host into which they are introduced; further, they should be capable of eliciting a protective immune response in that host. Included are viable herpes viruses that are incapable of replication (in the absence of an exogenous source of the protein, such as from a supporting host cell line expressing a complementing gene or genes) and are, therefore, incapable of generating progeny virus, but which is capable of expression of antigenic determinants such that a protective immune response is elicited.

Virus-specific products generally responsible for eliciting a protective immune response are proteins and glycoproteins that are expressed in the infected cell and, generally, are found on the surface of the virion. In the case of a herpes virus, some of the major antigenic determinants are glycoproteins encoded by the viral genome. Vaccine strains of the replication defective herpes virus can be produced that are capable of expressing either one or more proteins or glycoproteins normally expressed by an endogenous herpes virus or expressing a heterologous gene.

According to the claimed invention, the replication defective herpes virus can be administered to elicit a protective immune response or to enhance an immune response against a corresponding wild-type herpes virus. Alternatively, the replication defective herpes virus can be further genetically engineered, in accordance with known techniques, to express a heterologous protein. In one embodiment, the heterologous protein is an antigen that elicits a protective immune response or enhances an immune response against a corresponding heterologous wild-type pathogen. The heterologous gene or genes for the antigen or immunogen can be derived from another herpes virus, such as those enumerated above, or other infectious agents, such as viruses, bacteria, fungi or parasites. Preferably, the heterologous protein is an antigen of a human immunodeficiency virus (e.g., GAG polypeptide or an HIV envelope polypeptide), an antigen of a human papilloma virus, or any antigen expressed by a virus or bacteria associated with a sexually transmitted disease.

In one embodiment, the virus contains both HSV-2 and HSV-1 antigenic sequences. Such viruses are useful in protecting individuals against both HSV-1 and HSV-2 because they are capable of expressing antigenic determinants specific for both viruses. HSV-1 glycoprotein immunogens are disclosed by Sarmiento et al., J. Virol., 29:1159 (1979) (“gB”); Coker et al., J. Virol., 47:172-181 (1978) (“gD”); and DeSai et al., J. Gen. Virol., 69:1147-1156 (1988) (“gH”). These glycoprotein immunogens can be inserted in a mutated background, e.g., the genes encoding proteins required for replication in another herpes virus, as described above.

The invention also provides for a method of inducing an immunological response in an individual, particularly a human, which comprises inoculating the individual with a composition of the invention (e.g., virus, HSV-2 nucleic acid molecule, or infected cell), in a suitable carrier for the purpose of inducing an immune response to protect said individual from infection with an HSV-2 virus. The administration of this immunological composition may be used either therapeutically in individuals already experiencing an HSV-2 infection, or may be used prophylactically to prevent a herpes infection.

Therapeutic vaccines may reduce or alleviate a symptom associated with a herpes infection, such as the severity of a herpetic lesion. In some cases, a therapeutic vaccine will enhance the immune response of an individual infected with the virus. For example, the vaccines of the invention are useful for reducing the frequency or severity of symptomatic or asymptomatic herpetic outbreaks. Symptomatic outbreaks are characterized by the appearance of herpetic lesions or other clinical symptoms of infection. Asymptomatic outbreaks are characterized by viral shedding in the absence of herpetic lesions.

Prophylactic vaccines may be used to prevent or reduce the probability that a subject (e.g., a human) will be infected with the herpes virus. Most advantageously, a vaccine prevents the transmission of the virus from an infected individual to an uninfected individual. Also useful in the methods of the invention are vaccines that prevent the virus from establishing a latent infection in a herpes infected subject.

Also useful as therapeutic or prophylactic vaccines are cellular vaccines, which contain cells infected with an HSV-2 virus having a mutation in UL41, UL5, and/or UL29. Preferably, such vaccines include a cell (e.g., a dendritic cell) derived from the subject that requires vaccination. In general, the cell is obtained from a biological sample of the subject, such as a blood sample. Preferably, a dendritic cell or dendritic stem cell is obtained from the subject, and the cell is cultured in vitro to obtain a population of dendritic cells. The cultured cells are infected with a mutant HSV-2 virus containing a mutation in UL41, UL5, and/or UL29. The infected cells are then re-introduced into the subject where they enhance or elicit an immune response against a wild-type HSV-2 virus. Methods of making cellular vaccines are known in the art and are described, for example, by Garcia et al. (Journal of Infectious Diseases 191:1680-5, 2005) and by Babatz et al. (Cancer Immunol. Immunother. July 21; 1-9, 2005; Epub).

The preparation of vaccines is known to one skilled in the art. A virus or an expression vector encoding at least one viral polypeptide, or fragments or variants thereof, is delivered in vivo in order to induce an immunological response in a subject. Typically, the immune response includes the production of antibodies or a T cell immune response.

For example, an HSV-2 virus or nucleic acid molecule encoding a viral polypeptide is injected into a subject. In some embodiments, a viral polypeptide is fused to a recombinant protein that stabilizes the polypeptide and aids in its solubilization, facilitates its production or purification, or acts as an adjuvant by providing additional stimulation of the immune system. The compositions and methods comprising the polypeptides or nucleotides of the invention and immunostimulatory DNA sequences are publicly available. See, for example, the genome sequence of HSV-2 strain HG-52, which is provided at GenBank Accession No. Z86099.

Typically vaccines are prepared in an injectable form, either as a liquid solution or as a suspension. Solid forms suitable for injection may also be prepared as emulsions, or with the polypeptides encapsulated in liposomes. Vaccine antigens are usually combined with a pharmaceutically acceptable carrier, which includes any carrier that does not induce the production of antibodies harmful to the individual receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

Adjuvants are immunostimulating agents that enhance vaccine effectiveness. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Immunogenic compositions, i.e. the antigen, pharmaceutically acceptable carrier and adjuvant, also typically contain diluents, such as water, saline, glycerol, ethanol. Auxiliary substances may also be present, such as wetting or emulsifying agents, pH buffering substances, and the like. Proteins may be formulated into the vaccine as neutral or salt forms. The vaccines are typically administered parenterally, by injection; such injection may be either subcutaneous, intradermal or intramuscular. Additional formulations are suitable for other forms of administration, such as by suppository or orally. Oral compositions may be administered as a solution, suspension, tablet, pill, capsule, or sustained release formulation.

In addition, the vaccine can also be administered to individuals to generate polyclonal antibodies (purified or isolated from serum using standard methods) that may be used to passively immunize an individual. These polyclonal antibodies can also serve as immunochemical reagents.

In addition, it is possible to prepare live attenuated microorganism vaccines that express recombinant polypeptides, for example, HSV-2 antigens. Preferably, a live vaccine is replication defective or is incapable of reverting to a wild-type pathogenic phenotype.

Vaccines are administered in a manner compatible with the dose formulation. The immunogenic composition of the vaccine comprises an immunologically effective amount of a virus, cell, or antigenic nucleic acid molecule, and other previously mentioned components. By an immunologically effective amount is meant a single dose, or a vaccine administered in a multiple dose schedule, is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgment of the attending clinician.

### Induction or Enhancement of an Immune Response

Vaccines of the invention elicit a cellular immune response and/or a humoral immune response against a herpes virus (e.g., HSV-2). A humoral immune response includes the production of antibodies that specifically bind an HSV-2 antigen. Antibody molecules differ in their abilities to bind complement and Fc receptors. The functional properties of the IgG2a subclass Ig suggest that they are important in the defense against virus infection, in which opsonization, or enhanced attachment, and complement-mediated lysis of viruses and destruction of virus-infected cells by antibody-dependent cellular cytotoxicity are important. It is also the most effective subclass for the induction of macrophages and killer cells.

Interferon-gamma (IFN-gamma) is produced by helper T cells of the Th1 subdivision. IFN-gamma has diverse effects on a variety of cell types. It plays an important role in macrophage activation and has also been shown to affect polyclonal B cell activation and differentiation. IFN-gamma promotes the production of IgG2a by activated murine and human B cells stimulated in IL-2 and causes human B cells treated with antibodies against Ig to enter the S phase of the cell cycle.

Tumor necrosis factor-alpha (TNF-alpha), which is primarily released from macrophages, has been shown to display a synergistic effect with IFN-gamma in several functions (Lee et al., J. Immunol., 133:1083 (1984); Stone-Wolff et al., J. Exp. Med., 159:828 (1984); Williams et al., J. Immunol., 130:518 (1983)), including protection against lethal infection.

Preferably, an HSV vaccine of the invention is capable of eliciting or enhancing an immune response in a subject that is characterized by Th1 cell activation, IFN-gamma or other cytokine secretion, IgG production (e.g., IgG2a), TNFalpha production, or activation of cytotoxic T cells. Such vaccines are useful in treating a subject diagnosed as having an active or latent herpes infection, or of preventing a herpes virus infection in a subject at risk thereof.

In view of the ability of the replication defective herpes virus of the claimed invention to induce an immune response against a herpes virus and to increase production of HSV-2 IgG or IFN-gamma, the mutated herpes virus can be administered to treat a herpes virus infection or herpes-related condition in a subject (e.g., a herpetic lesion, herpetic stromal keratitis, meningitis, or encephalitis).

### Herpes Viral Vaccine Dosage

Those skilled in the art will understand that dosage can be optimized using standard procedures. In general, the vaccines and pharmaceutical compositions are formulated in suitable sterilized buffer and administered (e.g., by subcutaneous, intramuscular or intradermal injection) at a dosage of between 10³ and 10⁹ PFU/dose. The composition can also be administered by any known means successful in eliciting the immunomodulatory response and/or protective immune response, such as oral or ocular administration in vehicles known in the art.

### Combination Therapies

Compositions of the invention, such as a therapeutic vaccine for the treatment of a herpes infection may, if desired, be administered in combination with any antiviral therapy known in the art. Such therapies include, for example, treatment with acyclovir, valacyclovir, or famciclovir.

### Kits

The invention provides kits for the treatment or prevention of a herpes infection, including a herpetic lesion, herpetic stromal keratitis, meningitis, or encephalitis, or symptoms thereof. In one embodiment, the kit includes a therapeutic or prophylactic vaccine containing an effective amount of an HSV-2 virus having a mutation in UL41, a cell infected with such a virus, or an HSV-nucleic acid molecule comprising a mutation in UL41, in unit dosage form. In some embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired vaccines of the invention are provided together with instructions for administering them to a subject having or at risk of developing a herpes infection. The instructions will generally include information about the use of the vaccine for the treatment or prevention of a herpes infection. In other embodiments, the instructions include at least one of the following: description of the vaccine; dosage schedule and administration for treatment of a herpes infection or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The following examples are provided to illustrate the invention, not to limit it. Those skilled in the art will understand that the specific constructions provided below may be changed in numerous ways, consistent with the above described invention while retaining the critical properties of the vaccine or immunomodulant.

### EXAMPLES

### Replication Defective Mutant HSV Viruses

Replication defective mutant HSV viruses have been used for the production of therapeutic and prophylactic vaccines useful for treatment of herpes infections in infected individuals as well as for preventing herpes infections in susceptible individuals or preventing the emergence of active lesions in chronically infected individuals. The HSV-2 dl5-29 replication defective mutant virus retains the ability to infect cells and produce viral proteins, but lacks the ability to generate infectious viral progeny. The dl5-29 virus contains deletions in each of two genes, UL5 and UL29 (ICP8). The UL5 gene encodes a component of the helicase/primase complex. The UL29 gene encodes an ssDNA binding protein. Each of these genes is essential for DNA replication. dl5-29 has been shown to induce protective immunity in both mice (DaCosta, X., et. al. PNAS USA. 96, 6994, 1999) and in guinea pigs (Hoshino, Y., et. al. J. Virol. 79, 410, 2005). As reported below, dl5-29 also induces protects against infection with SD90, a virulent South African clinical isolate of HSV-2.

In general, the present invention is based on the discovery that a replication defective HSV-2 virus having a mutation in UL41, the gene that encodes the virion host shut-off protein (VHS), is more effective in eliciting a host immune response than a corresponding HSV-2 virus containing a wild-type UL41 gene.

### Example 1

### dl5-29 Immunization Induces Protection Against a Virulent HSV-2 Isolate

To determine if dl5-29 can induce protection against a primary clinical isolate from Sub-Saharan Africa, a highly virulent South African HSV-2 genital isolate, SD90, was identified. Mice were immunized twice with dl5-29 and then challenged intravaginally with 50 times the LD₅₀ of either the SD-90 isolate or the SD90-3P purified clone. dl5-29 was able to elicit protection with 100% survival against this high dose of either the virulent HSV-2 isolate or the clone from Sub-Saharan Africa.

### Example 2

### Disruption of the UL41 Gene Enhances the Humoral Immune Response

The HSV-2 UL41 gene encodes the virion host shut-off (VHS) polypeptide. VHS enhances herpes virulence and pathogenicity by inhibiting a number of host biological functions that are required to generate a robust host immune responses. VHS causes the nonspecific degradation of host and viral mRNA during early stage herpes infections. In addition, VHS is a potent inhibitor of the IFN-mediated anti-viral response and is responsible for HSV-mediated blocking of dendritic cell activation.

Deletion of the UL41 virion host shut-off (vhs) gene has been shown to increase the immunogenicity of HSV-1 replication defective viruses (Geiss, B., et. al. J. Virol. 74, 11137). To evaluate the effect of this gene on the immunogenicity of HSV-2 replication defective viruses, a LacZ insertion into UL41 was generated in the dl5-29 HSV-2 virus. The HSV-2 virus containing the triple mutant was designated dl5-29-41LacZ. The ability of the triple mutant dl5-29-41LacZ to elicit an immune response was compared to that of dl5-29.

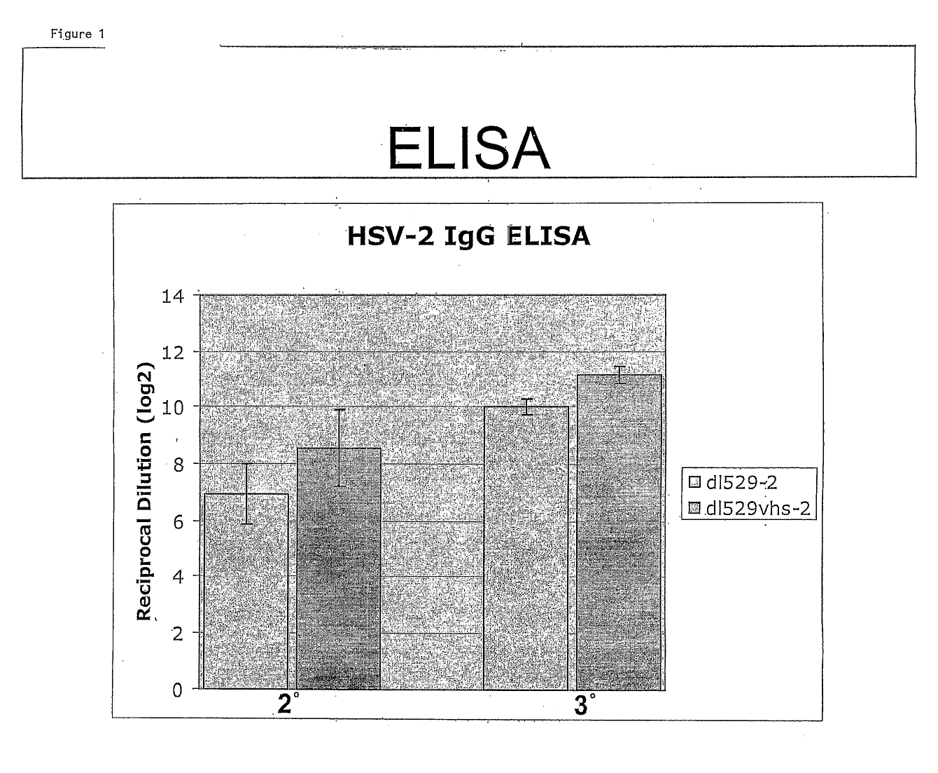

To evaluate the humoral immune response, mice were vaccinated with one, two or three doses of either a double deletion dl5-29 vaccine or a triple mutant dl5-29-41LacZ vaccine and the total IgG levels were analyzed seven days post-vaccination. As shown in FIG. 1, mice immunized with the triple mutant dl5-29-41LacZ vaccine generated a significantly more robust immune response (FIG. 1).

### Example 3

### Deletion of the UL41 Gene Enhances the Cellular Immune Response

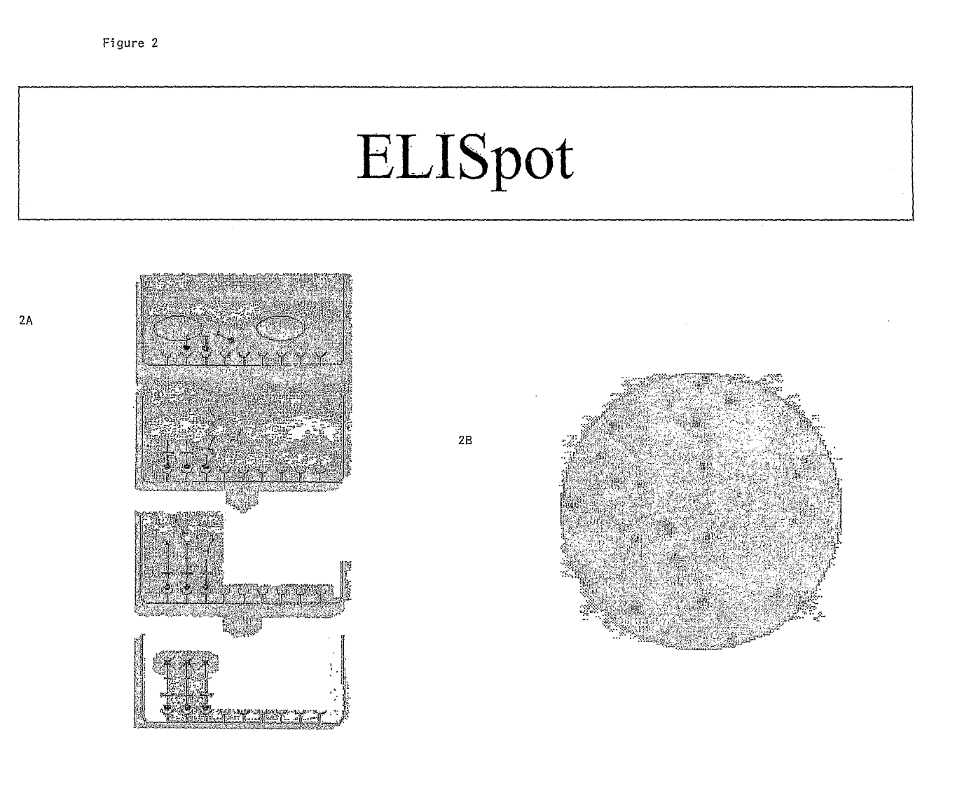

The cellular immune response of C57 Bl/6 mice was evaluated using an enzyme-linked immunospot assay (ELISpot), which provides for the high resolution frequency analysis of IFN-γ-secreting cells and the visualization of secretory products of individual cells. FIG. 2A is a schematic diagram illustrating the ELISpot assay. As shown in FIGS. 2A and 2B, isolated splenocytes are plated onto ELISpot plates, which have IFN-gamma specific antibodies fixed to their surface. The cells are then treated with an HSV gB peptide (SSIEFARL). This peptide specifically binds an MHC-1 molecule that is expressed on cd8(+) T cells. Only splenocytes that recognize this peptide secrete IFN-gamma. The secreted IFN-gamma binds an anti-IFN-gamma antibody that is fixed to the surface of the plate. The IFN-antibody is recognized by an avidin-coupled anti-mouse secondary antibody, which is visualized with streptavidin horseradish peroxidase. FIG. 2B illustrates exemplary ELISpot results. ELISpot measures the frequency of cells in a population that generate an HSV-2 specific response.

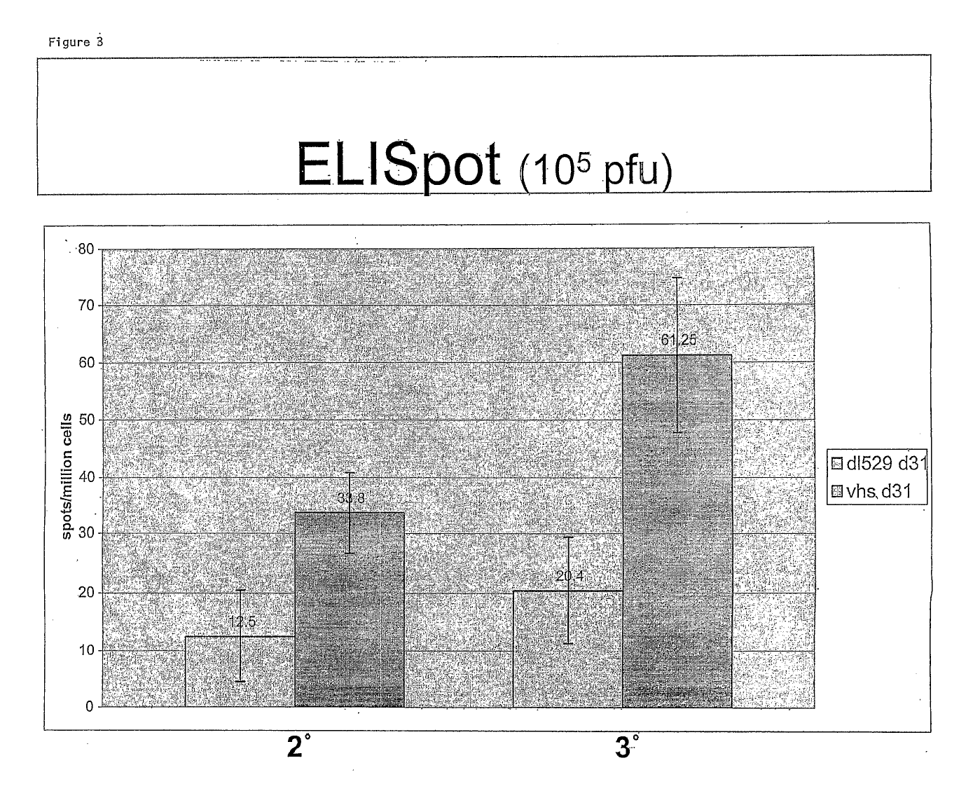

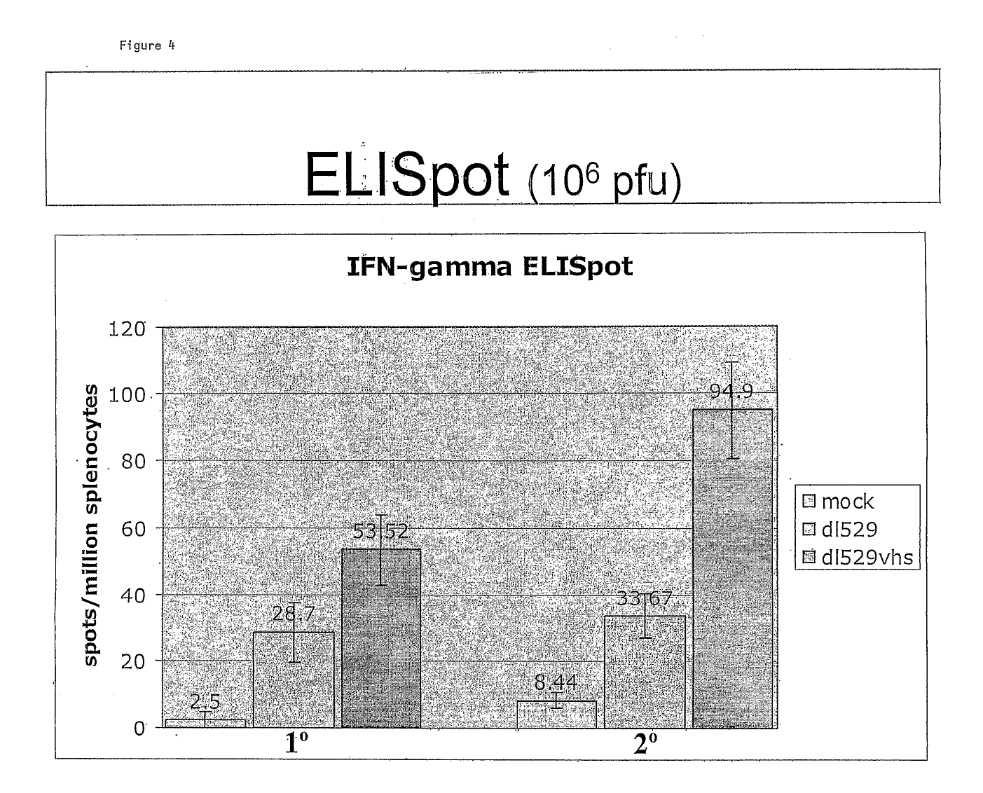

When the murine immune response is assayed using ELIspot, striking differences in the immunogenicity of the double and triple mutant viruses are observed in the response of isolated splenocytes to HSV gB peptide. Mice immunized with 10⁵ pfu (FIG. 3) or 10⁶ pfu of the triple mutant dl5-29-41LacZ virus (FIG. 4) show a more robust cellular immune response after primary or secondary vaccination than mice that received the double mutant dl5-29 vaccine. In fact, the cellular immune response in mice that received vaccines containing the dl5-29-41LacZ virus was 2-3 fold higher than that observed in mice that received the dl5-29 virus (FIGS. 3 and 4).

### Example 4

### dl5-29-41LacZ Provides Greater Protection Against HSV-2 G Challenge than dl5-29

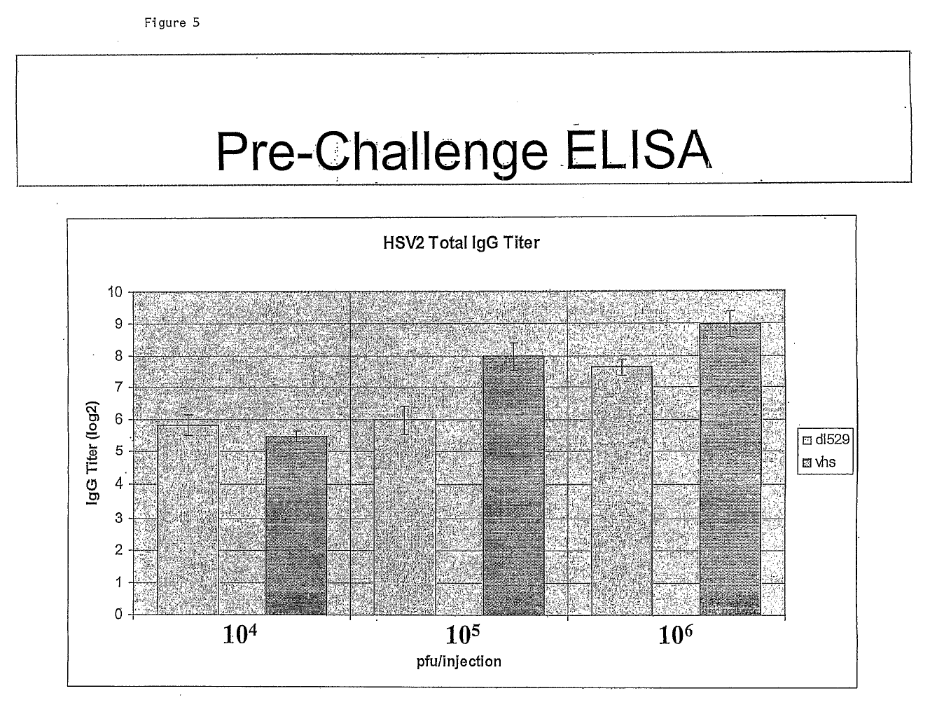

Balb/c mice were vaccinated with 10⁴, 10⁵, or 10⁶ pfu of dl5-29-41LacZ or dl5-29 mutant virus using the immunization schedule described above. The HSV total IgG titer present in the serum from these mice was determined at day seven post-vaccination. dl5-29-41LacZ elicited a significantly stronger humoral immune response than dl5-29 as measured by ELISA (FIG. 5). In fact, HSV2 total IgG titer was 3-4 fold higher in mice that received the dl5-29-41LacZ vaccine relative to mice that received the dl5-29 vaccine.

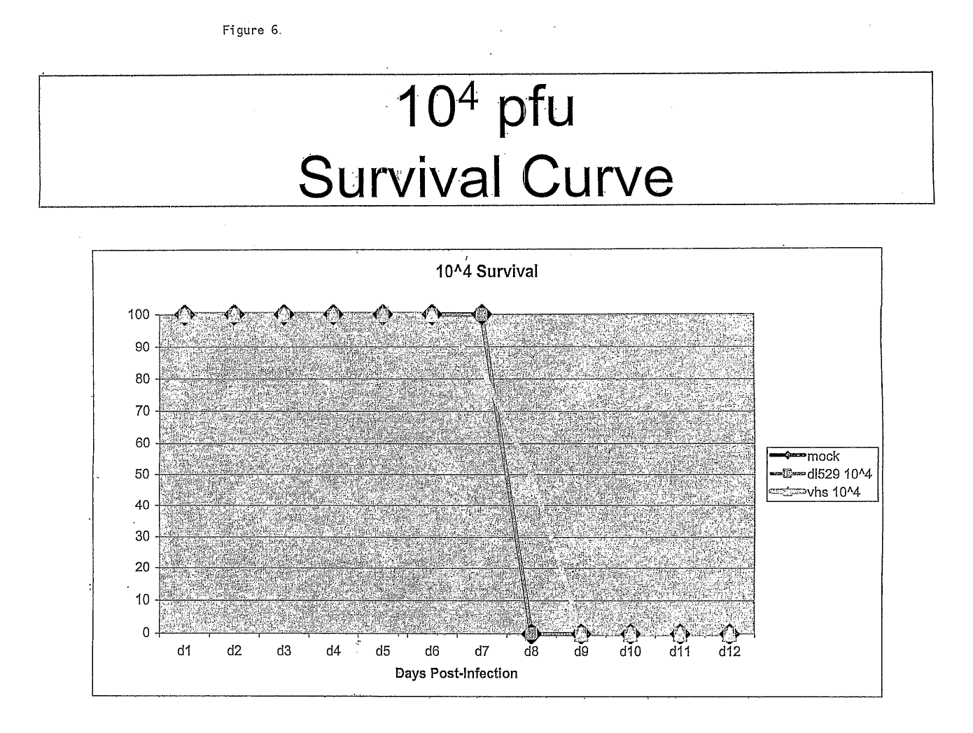

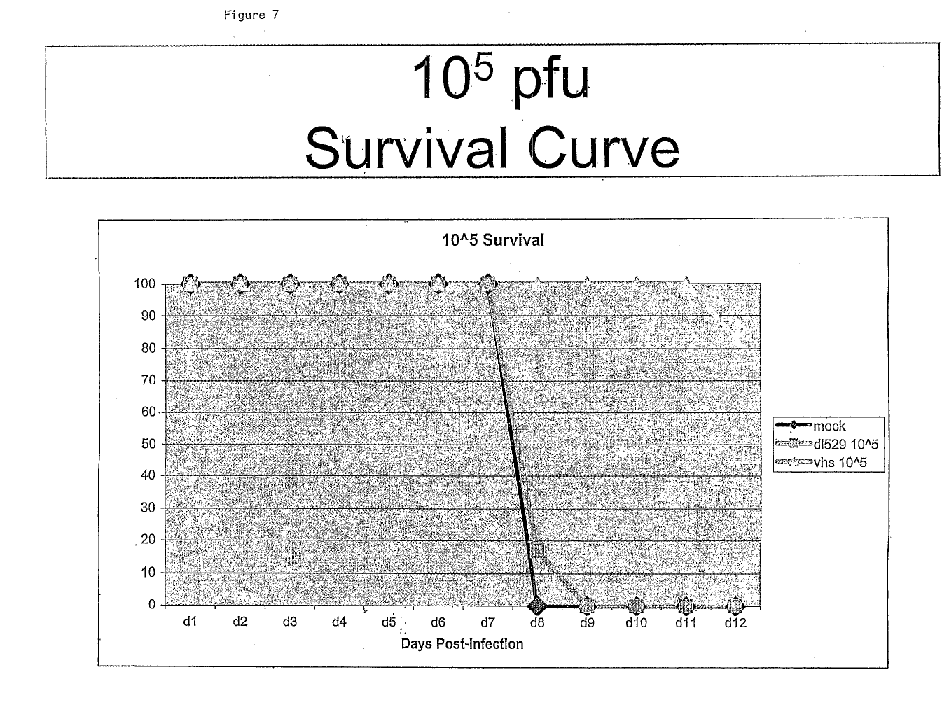

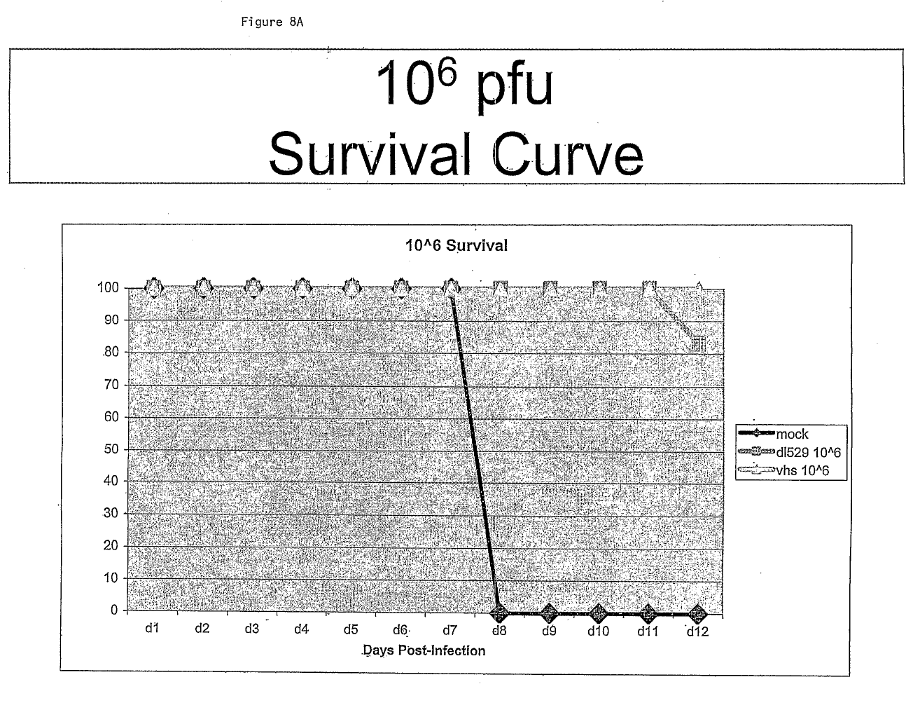

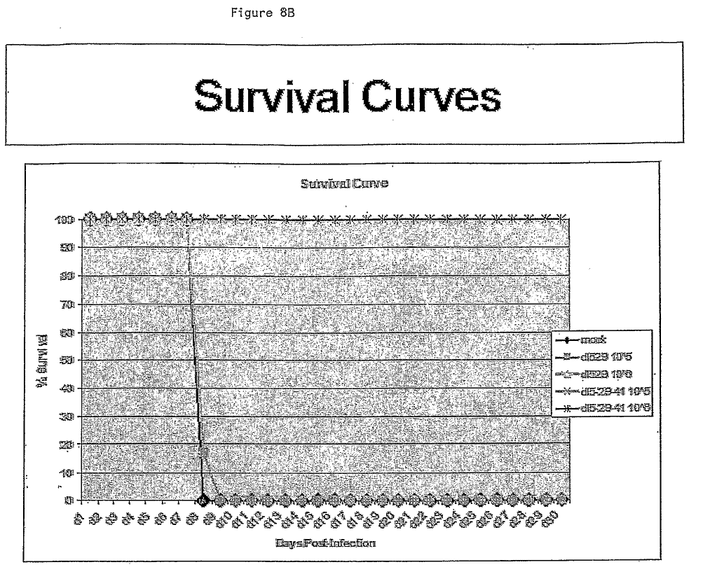

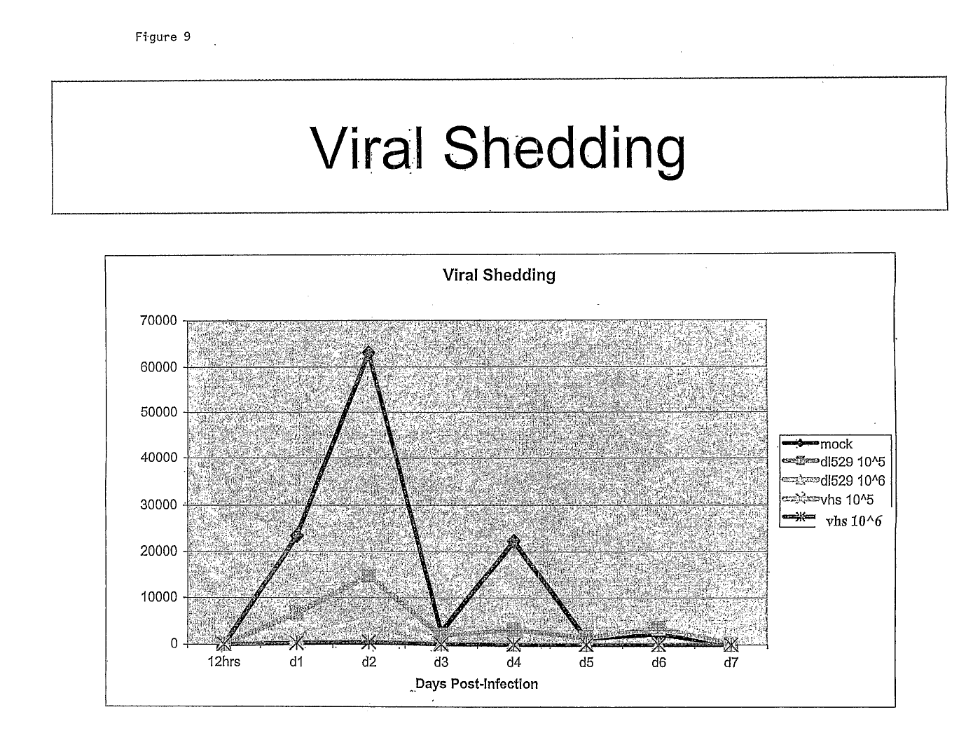

The vaccinated Balb/c mice were subsequently challenged with a highly lethal dose of HSV-2 strain G. The protective efficacy of the vaccine was then measured by assaying survival and viral shedding. As shown in FIGS. 6, 7, 8A and 8B, mice immunized with a triple mutant dl5-29-41LacZ vaccine survived significantly longer than mice that received a double deletion dl5-29 vaccine. Mice that received the triple mutant dl5-29-41LacZ vaccine also exhibited less viral shedding than mice that received the double deletion dl5-29 vaccine (FIG. 9). One important difference seen between dl5-29 and dl5-29-41LacZ is in the survival curve comparing dl5-29 at 10⁵ pfu and 10⁶ vaccination doses and dl5-29-41LacZ at 10⁵ vaccination dose. This data shows that the effective dose needed to protect 50% of the mice (PD50) is greater than 10× less with dl5-29-41LacZ than with dl5-29.

| Vaccine | PD50 |
|---|---|
| dl5-29 | 5.62 × 10⁵ |
| dl5-29-41LacZ | 3.98 × 10⁴ |

In fact, the triple mutant dl5-29-41LacZ showed greater than a 10-fold increase in protective efficacy against a highly lethal HSV-2 G challenge relative to the double deletion dl5-29.

### Example 5

### Cells Infected with DL5-29-41LacZ Produce Higher Levels of Viral Proteins than Cells Infected with DL5-29

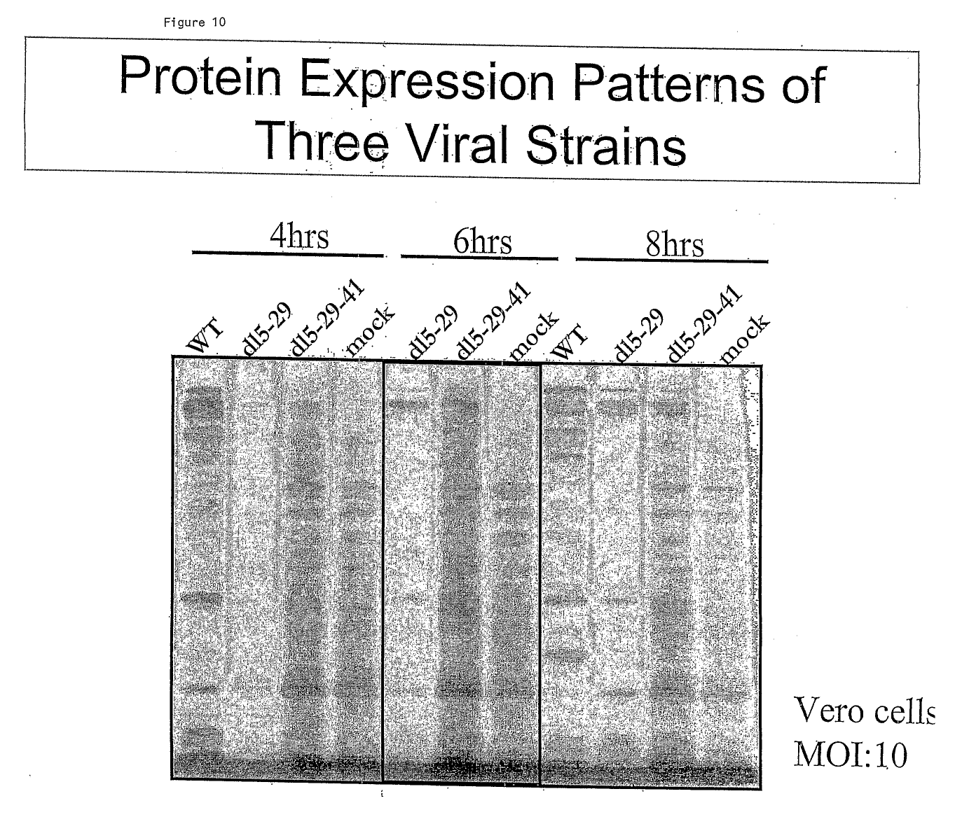

Vero cells were infected with wild-type virus, dl5-29, dl5-29-41LacZ, or were mock infected. The infected cells were cultured in methionine and cysteine deficient media that was supplemented with ³⁵S labeled methionine and cysteine. Viral protein expression was then assayed. The results of these studies are shown in FIG. 10. Viral proteins can be seen as the darker bands found in all lanes but the mock infected cell. Host cell protein expression levels can be estimated by the level of darkness or amount of background signal seen in each lane. The expression levels of viral proteins are similar between dl5-29 and dl5-29-41LacZ infected cells, while the levels of host cell expression is reduced in cells infected with dl5-29. Cells infected with dl5-29-41LacZ have host cell protein expression levels similar to that of uninfected cells, indicating a loss of the ability to inhibit host cell protein expression.

In sum, a replication defective herpes virus lacking VHS, dl5-29-41LacZ, was significantly more effective in generating an immune response in mice than was a replication defective virus expressing the VHS protein. Without being tied to one particular theory, the improved efficacy of the virus is likely attributable to several factors; first, the dl5-29-41LacZ virus likely activated dendritic cells more effectively than the double mutant; second, the DL5-29-41LacZ virus was likely less cytopathic than the DL5-29 deletion; and third, the dl5-29-41LacZ did not shut-down host protein production as the DL5-29 virus did. Because host cells infected with the dl5-29-41LacZ virus survived longer, immune cells were more likely to recognize the presence of an infection. In addition, because host protein synthesis was not shut down, the host was capable of generating a more effective immune response.

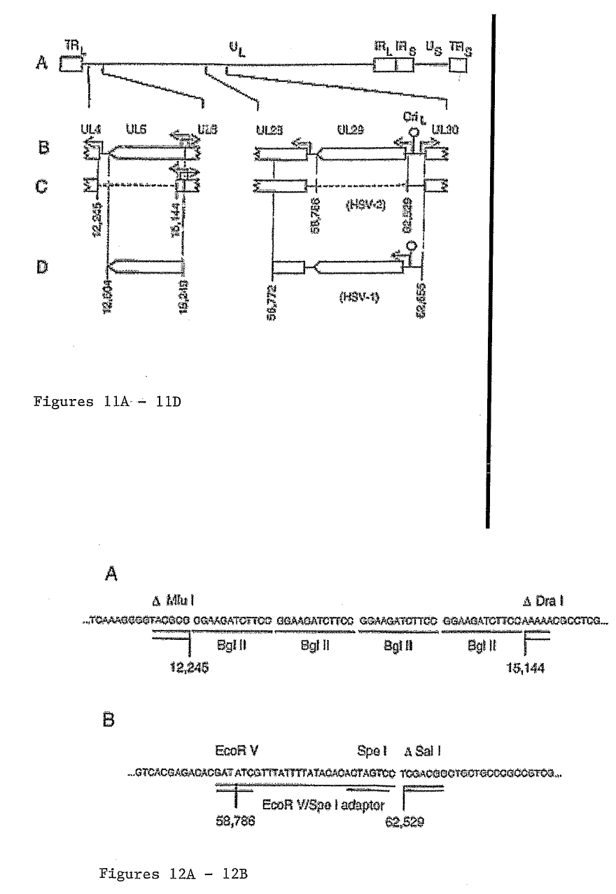

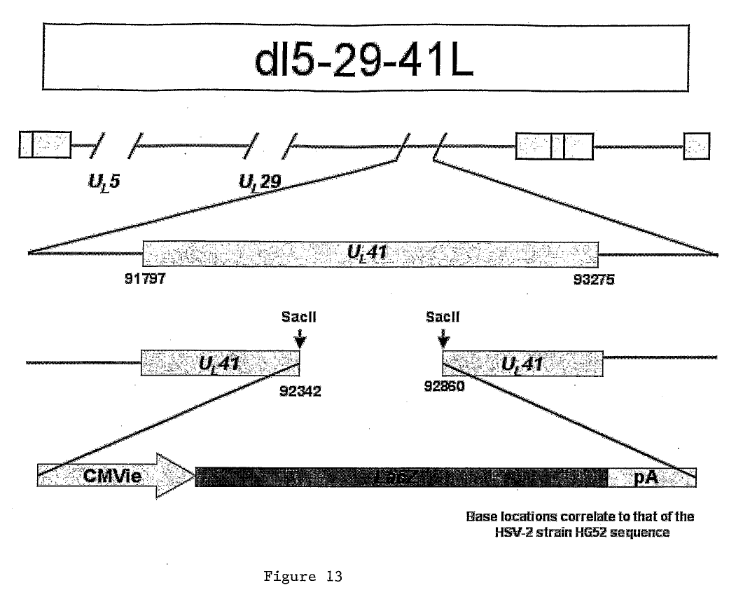

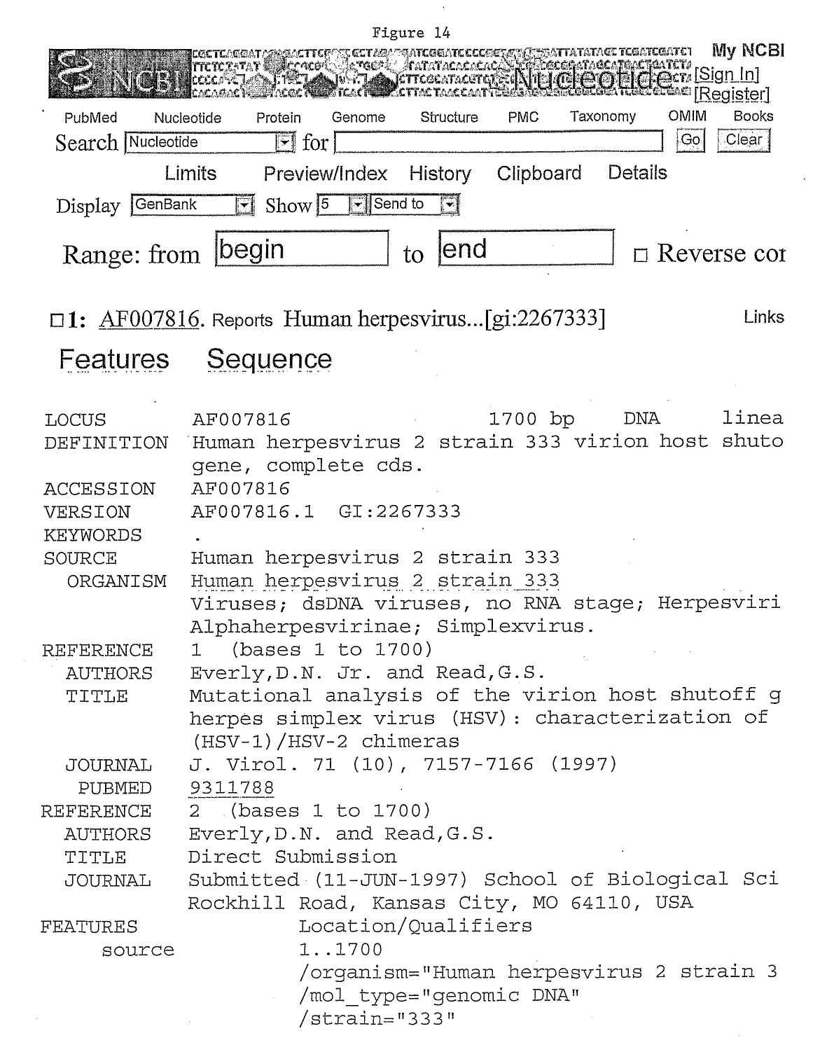

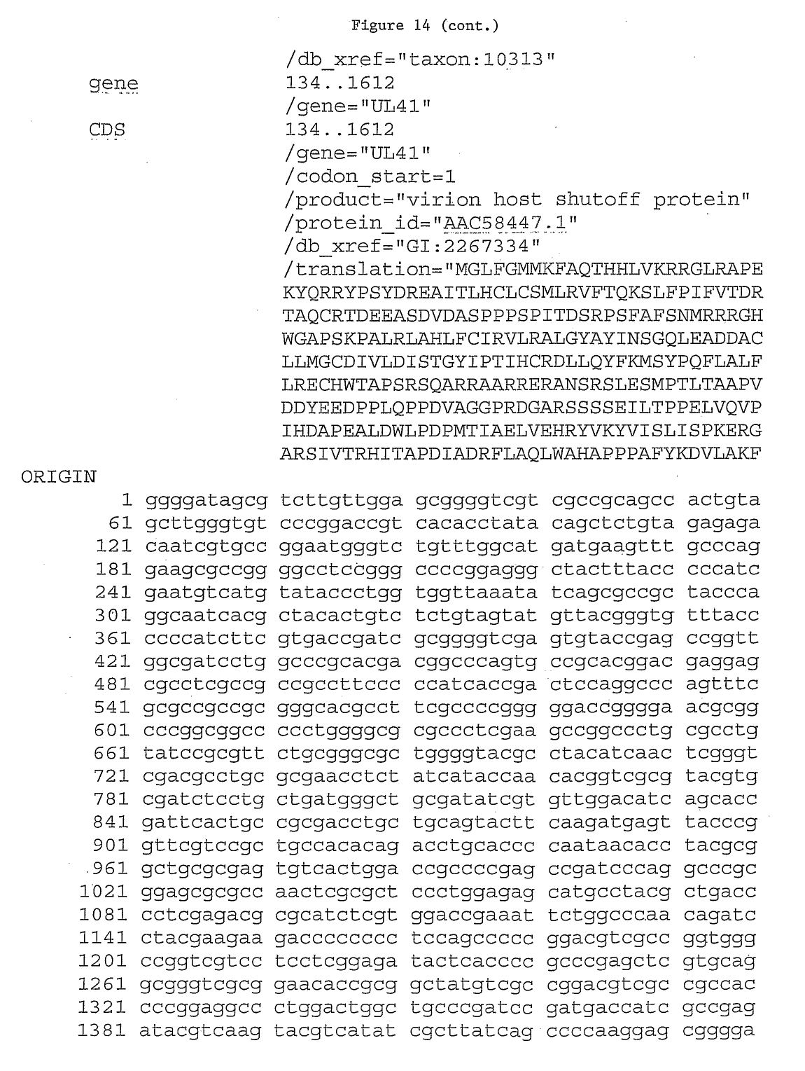

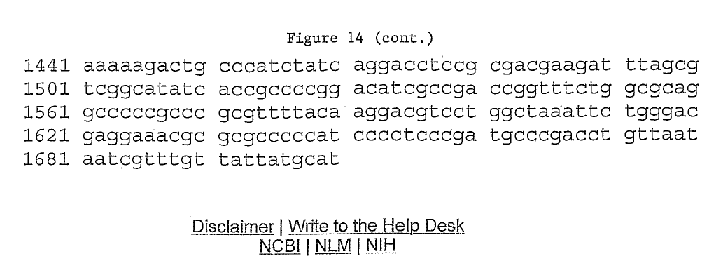

Schematic diagrams showing the genome structure and location of the mutations in UL5 and UL29 are provided at FIGS. 11A-11D. Sequence of the dl5 and dl29 deletion sites is provided at FIGS. 12A and 12B. A schematic diagram showing the base location of the LacZ insertion in UL41 is provided at FIG. 13. The UL41 gene extends from HG-52 HSV2 genome base number 91797 to 93275. The LacZ insertion in UL41 extends from 92342 to 92860. The sequence of a wild-type UL41 gene is shown in FIG. 14.

These experiments were carried out using the following methods and materials.

### Cells.

Vero (American Type Culture Collection [ATCC], Manassas, Va.) and Vero-derived cell lines were grown and maintained in Dulbecco's modified Eagle's medium supplemented with 10% fetal bovine serum (FBS).

### Animals and Animal Protocols.

Female BALB/c mice were used for HSV-2 challenge assays. BALB/c mice were obtained from Taconic Farms (Germantown, N.Y.) and housed in accordance with institutional and National Institutes of Health guidelines on the care and use of animals in research. C57 Bl6 mice were used for ELISA and ELIspot assays. Immunization of mice was performed as described previously (J. Virol. 2000; 74: 7699-8218). In brief, six-week-old female BALB/c or B57 Bl/6 mice were randomized into four groups of six mice each. All animals were injected two or three times, 3 weeks apart, in the hind flank by the subcutaneous route with 10⁴, 10⁵, or 10⁶ PFU of dl5-29 or dl5-29-41LacZ. Mice were bled from the tail vein seven days after the primary inoculation and a second time seven days after the boost inoculations. Serum was isolated using Becton Dickinson Microtainer serum separator tubes (VWR, Boston, Mass.) and stored at −20° C.

### Analysis of Viral Proteins

Levels of gene expression in virus- and mock-infected cells were examined as described previously (J. Virol. 2000; 74: 7699-8218). Briefly, cells were infected with the indicated virus or were mock infected and then incubated in medium 199 with 1% calf serum. At the indicated times, cells were overlaid with 2 ml of labeling medium (Eagle's minimum essential medium, without methionine or cysteine [Sigma, St. Louis, Mo.], containing 100 μCi of EXPRE³⁵S³⁵S™ protein labeling mix with [³⁵S]methionine and [³⁵S]cysteine [NEN] and supplemented with 10% dialyzed FBS [GIBCO/BRL, Gaithersburg, Md.]) for 30 minutes. Cells were solubilized with 1.0 ml of gel sample buffer (0.5 M Tris-HCl [pH 6.8], 20% glycerol, 20% sodium dodecyl sulfate [SDS], 5% β-mercaptoethanol, 0.5% bromphenol blue) which also contained 50 μg of N-p-tosyl-L-lysine chloromethyl ketone (TLCK) per ml. Proteins were resolved by electrophoresis in a 9.25% N—N′ diallyltartardiamide (DATD)-cross-linked polyacrylamide gel. The gels were dried under vacuum and exposed to Biomax MR single-sided emulsion film.

### Splenocyte Isolation

Spleens are aspetically removed and physically disrupted to obtain a single cell suspension in ice cold PBS. Cells are then pushed through a 100 um nylon cell strainer (BD Biosciences, Beford, Mass.) two times. Splenocytes are then washed in ice cold PBS three times, counted and aliquoted for use in the ELISpot Assay.

### ELISPOT Assay

ELISpot assays were performed using an enzyme-linked immunospot assay kit, the Mouse IFN-gamma ELISpot Set (BD Biosciences, San Diego, Calif.). BD Elispot plates were equilibrated with PBS and coated with a purified anti-mouse IFN-gamma capture antibody (BD Biosciences) overnight at 4° C. Plates were then blocked with 200 ul complete media (DMEM, 10% FCS, 1% Pen/Strep) for two hours at room temperature. Blocking media was then removed and splenocytes were added at 2×10⁵ cells/well and 5×10⁴ cells/well in 200 ul complete media containing IL-2 (100 U/ml). Experimental wells were stimulated with the H2 Kb specific HSV gB peptide SSIEFARL (SEQ ID NO:1) (10 ng/ml). Negative controls were stimulated with the H2 Kb specific OVA peptide SIINFEKL (SEQ ID NO:2) (10 ng/ml). Plates were incubated at 37° C. in a 5% CO₂ humidified incubator overnight. Cells were aspirated and wells washed three times with deionized water, followed by two washes with PBS containing 0.05% Tween-20. Biotinylated anti-mouse IFN-gamma detection antibody (BDBiosciences) was added to each well in 100 ul at 2 ug/ml. Plates were incubated for two hours at room temperature. Plates were washed three times with PBS 0.05% Tween-20. 100 ul Strepavidin-HRP enzyme substrate was added to each well at 50 ug/ml and incubated for one hour at room temperature. Plates were washed three times with PBS 0.05% Tween-20 followed by two washes with deionized water. Spots were developed using 3-amino-9-ethyl carbazole (AEC) in a commercially available AEC substrate mix (BD Biosciences, Mountain View, Calif.) at room temperature for ten minutes.

### ELISA

Ninety six-well high absorption, MaxiSorb plates (Nalge NUNC, Rochester N.Y.) were coated with HSV-2 antigen (Advanced Biotechnologies Inc., Columbia, Md.) at 50 ng per well in 0.05 M carbonate-bicarbonate buffer (pH 9.6) (Sigma) overnight at 4° C. Plates were blocked with PBS containing 5% milk at 37° C. for 1 hour, washed three times with PBS containing 0.05% Tween 20, and incubated with serial twofold dilutions of mouse sera for two hours at 37° C. Plates were then washed three times with PBS containing 0.05% Tween 20 and incubated with goat anti-mouse IgG antibody conjugated with alkaline phosphatase (1:1,000; Sigma) for 1 hour at 37° C. Finally, plates were washed three times with PBS containing 0.1% Tween 20, developed using SigmaFast p-nitrophenyl phosphate (Sigma St. Louis, Mo.), and read at 405 nm. Antibody titers indicate the final reciprocal dilution resulted in optical density readings greater than 0.2 unit above background. Results are expressed as the geometric mean (log₂)±standard deviation, where log₂ 6=1:64 and log₂ 12=1:4,096. Enzyme-linked immunosorbent assay (ELISA) results were analyzed for statistical significance by Student's t test.

Challenge with 50LD50 HSV-2 G

Seven days after the final immunization and 1 day prior to challenge, mice were injected subcutaneously in the scruff of the neck with depoprovera (3 mg/mouse in 100 ul). Three weeks after the final immunization intravaginal challenge was performed by inoculating 5×10⁵ pfu of HSV-2 strain G (50LD₅₀) in a 15 ul volume into the vaginal cavity using a micropipettor tip.

### Analysis of Viral Shedding

At 12 hours post-challenge and then once every day thereafter for ten days the vaginal mucosa of mice were swabbed twice and swabs were placed into a vial containing 1 ml of PBS 1% fetal calf serum, 0.1% glucose and stored at −80° C. To determine viral titers, vials were thawed, vortexed, serially diluted and inoculated onto a monolayer of Vero cells at 37° C. for 1 hour. Inoculum was removed and cells were incubated at 37° C. in a 5% CO₂ humidified incubator for three days in DMEM 1% FCS, 1% Pen/Strep containing 2 ug/ml of human sera. After three days cells were fixed with methanol and stained with Geimsa. Individual plaques were counted to determine the number of pfu/ml of virus being shed.

### Other Embodiments

From the foregoing description, it will be apparent that variations and modifications may be made to the invention described herein to adopt it to various usages and conditions. Such embodiments are also within the scope of the following claims.

All patents and publications mentioned in this specification are herein incorporated by reference to the same extent as if each independent patent and publication was specifically and individually indicated to be incorporated by reference.

1. An isolated herpes simplex virus 2 (HSV-2) comprising an alteration in a UL41 nucleic acid sequence, wherein the alteration increases the immunogenicity of the virus, and at least one additional alteration in a nucleic acid sequence required for viral replication.

2. The isolated HSV-2 virus of claim 1, wherein the alteration in UL41 is
a missense mutation, insertion or deletion in UL41 or
an insertion in UL41 that interferes with the expression of a virion host shut-off protein.

3. (canceled)

4. The isolated HSV-2 virus of claim 2, wherein the alteration in UL41 is the deletion of at least a portion of UL41.

5. The isolated HSV-2 virus of claim 4, wherein the alteration is the deletion of UL41.

6. The isolated HSV-2 virus of claim 1, wherein the additional alteration is a missense mutation, insertion or deletion in a UL5 or UL29 nucleic acid sequence.

7. (canceled)

8. The isolated HSV-2 virus of claim 6, wherein the additional alteration renders the virus replication defective.

9. The isolated HSV-2 virus of claim 8, wherein the additional alteration is the deletion of UL5 and UL29.

10. A host cell comprising the virus of claim 1.

11-12. (canceled)

13. The host cell of claim 10, wherein the mammalian cell is selected from the group consisting of a dendritic cell or other professional antigen presenting cell.

14. An isolated HSV-2 nucleic acid molecule comprising an alteration in a UL41 nucleic acid sequence that is a missense mutation, insertion or deletion in UL41 or an insertion in UL41 that interferes with the expression of a virion host shut-off protein and at least one additional alteration in a UL5 or UL29 nucleic acid sequence, wherein the additional alteration is a missense mutation, insertion or deletion.

15-26. (canceled)

27. A vector comprising the nucleic acid molecule of claim 14.

28. The vector of claim 27, wherein the vector is a viral vector.

29. The vector of claim 28, wherein the viral vector is selected from the group consisting of adenoviral vectors, adeno-associated viral vectors, retroviral vectors, lentiviral vectors, alphaviral vectors, and herpes virus vectors.

30. A host cell comprising the vector of claim 27.

31. (canceled)

32. A vaccine comprising an HSV-2 virus or a cell infected with said virus in a pharmaceutically acceptable excipient, wherein the virus comprises an alteration in a nucleic acid sequence that encodes a virion host shut-off protein or an alteration in a UL41 nucleic acid sequence and an additional alteration that renders it replication defective.

33-42. (canceled)

43. The vaccine of claim 32, wherein the composition elicits a cellular or humoral immune response when injected into a subject.

44-57. (canceled)

58. The vaccine of claim 32, wherein the cell is a dendritic cell.

59. The vaccine of claim 58, wherein the cell is cultured and infected ex vivo.

60-72. (canceled)

73. A method for treating or preventing a herpes infection in a subject, the method comprising administering to the subject a vaccine of claim 32, wherein the vaccine treats or prevents a herpes infection.

74-85. (canceled)

86. A kit for treating or preventing a herpes viral infection in a subject, the kit comprising a herpes vaccine of claim 32 and instructions for administering the herpes virus to a subject.

87-88. (canceled)
